(19) United States
(12) Patent Application Publication
(10) Pub. No.: US 2007/0208318 A1
(43) Pub. Date: Sep. 6, 2007

(54) Disposable pant-like absorbent undergarment closely resembling cloth underwear

(75) Inventors: Jeffrey James Loritz, Appleton, WI (US); Stacy Nicole Markovich, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/367,219

(22) Filed: Mar. 3, 2006

(51) Int. Cl. A61F 13/15

(52) U.S. Cl. 604/385.22; 604/385.24

(57) ABSTRACT

A disposable pant-like absorbent undergarment is disclosed having a chassis with stretchable front and back regions bridged by a crotch region. The front region has a first end and the back region has a second end and the chassis is folded such that the two ends are aligned. A waistband is secured to the first and second ends and projects outward therefrom. A pair of side seams join the front and back regions together and extend through the waistband to form an undergarment having a longitudinal central axis, a waist opening and a pair of leg openings. Elastic members at least partially surround the pair of leg openings. The waistband has a greater retracted length than the elastic members and the waistband imparts a convex configuration to each of the pair of side seams before the undergarment is worn by a user.

### BACKGROUND OF THE INVENTION

A disposable pant-like absorbent undergarment is designed for absorbing human exudate and has a waist opening and a pair of leg openings. The disposable pant-like absorbent undergarment tries to approach the appearance, size and shape of regular cloth underwear. The disposable pant-like absorbent undergarment differs from regular cloth underwear in that it is not designed to be laundered and reused two or more times. A disposable pant-like absorbent undergarment is intended to be worn by persons, including infants, toddlers or adults and is designed for single or temporary use. The disposable pant-like absorbent undergarment is meant to be disposed of after being used once instead of being cleaned and reused. The disposable pant-like absorbent undergarment is designed to be pulled up around the user's torso without having to first open the undergarment in order to place it on a user's body. The stretchability of the material used to construct the disposable pant-like absorbent undergarment permits the undergarment to snugly conform to the anatomy of the user's torso. The disposable pant-like absorbent undergarment can be manufactured to be an infant diaper, a child training pant, an adult incontinent garment, a feminine menstrual pant, etc.

Today, manufacturers are being asked by consumers to design disposable pant-like absorbent undergarments that more closely resemble cloth underwear. This is especially true for adults who are just starting to wear incontinent undergarments. Most disposable pant-like absorbent undergarments when initially removed from a package have a front profile that appears to be square or boxy. This is especially true of the spaced apart side seams that are aligned parallel to one another. Another difference is that most disposable pant-like absorbent undergarments are constructed using polyolefin materials or stretch bonded laminates that do not feel as soft as cotton. In fact, some materials used to form the outer cover on disposable pant-like absorbent undergarments have a rather stiff and inflexible feel to them. A third difference is that many disposable pant-like absorbent undergarments utilize either an integral waistband or use a plurality of elastic strands located between an inner and an outer cover to create a tensioned area that is several inches in width. This differs from cloth underwear that normally employs a distinguishable waistband which exhibits a more aesthetically pleasing appearance.

Now a disposable pant-like absorbent undergarment for absorbing human discharge has been invented that closer resembles cloth underwear.

### SUMMARY OF THE INVENTION

Briefly, this invention relates to a disposable pant-like absorbent undergarment having a chassis constructed from a stretchable front region, a stretchable back region, and bridged by a crotch region. The front region has a first end and the back region has a second end and the chassis is folded such that the first end aligns with said second end. An absorbent assembly is present in the crotch region. A waistband is secured to the first and second ends and projects outward therefrom. A pair of side seams join the front and back regions together and extend through the waistband to form a pant-like undergarment having a longitudinal central axis, a waist opening and a pair of leg openings. Elastic members at least partially surround the pair of leg openings, wherein the waistband has a greater retracted length than the elastic members. In addition, the elastic members have a greater retracted length than the chassis located between the waistband and the elastic members. Furthermore, the waistband imparts a convex configuration to each of the pair of side seams relative to the longitudinal central axis before the undergarment is worn by a user.

### BRIEF DESCRIPTION OF THE DRAWINGS

FIG. 1 is a front view of a disposable pant-like absorbent undergarment having a stretchable chassis with a waistband surrounding a waist opening and having a pair of leg openings at least partially surrounded by elastic members.

FIG. 2 is a plane view of the disposable pant-like absorbent undergarment shown in FIG. 1 depicting an absorbent secured to an inner surface of the stretchable front and back regions and showing two partial cut away views of the construction of the absorbent assembly and the elastic members.

FIG. 3 is a side view of the disposable pant-like absorbent undergarment shown in FIG. 2 and taken along line 3-3.

FIG. 4 is an enlarged view showing an alternative embodiment wherein the waistband is secured to an inner surface of the front region.

FIG. 5 is an enlarged view showing still another alternative embodiment wherein the front region is constructed of a two-layer laminate and the waistband is secured between the two layers.

### DETAILED DESCRIPTION

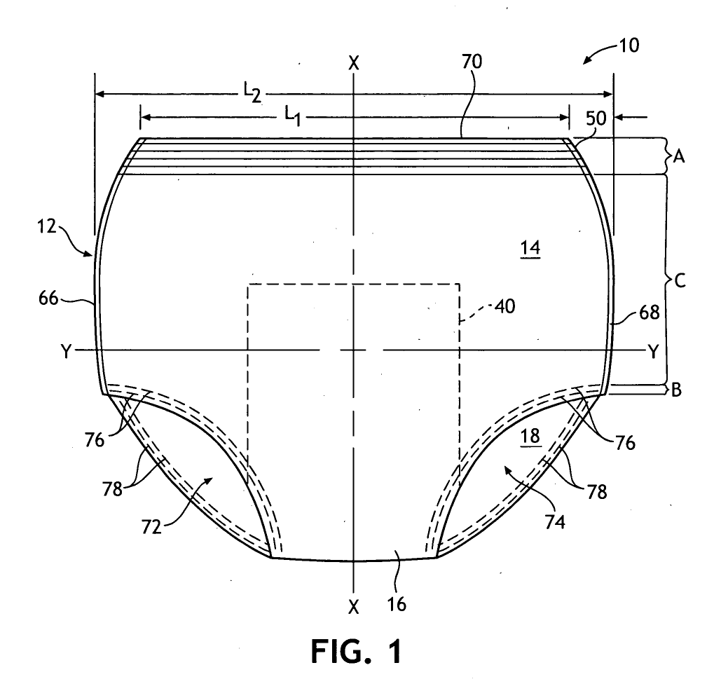

Referring to FIG. 1, a disposable pant-like absorbent undergarment 10 is depicted. The disposable pant-like absorbent undergarment 10 is intended to be worn by persons, including infants, toddlers or adults and is designed for a single or temporary use. The disposable pant-like absorbent undergarment 10 is meant to be disposed of after being used once instead of being laundered or cleaned for reuse. The disposable pant-like absorbent undergarment 10 is designed to be pulled up around a user's torso without having to first open the undergarment 10 in order to place it on a user's body. In FIG. 1, the disposable pant-like absorbent undergarment 10 is shown as it would appear after it has been removed from the package but before it is pulled up around a user's torso.

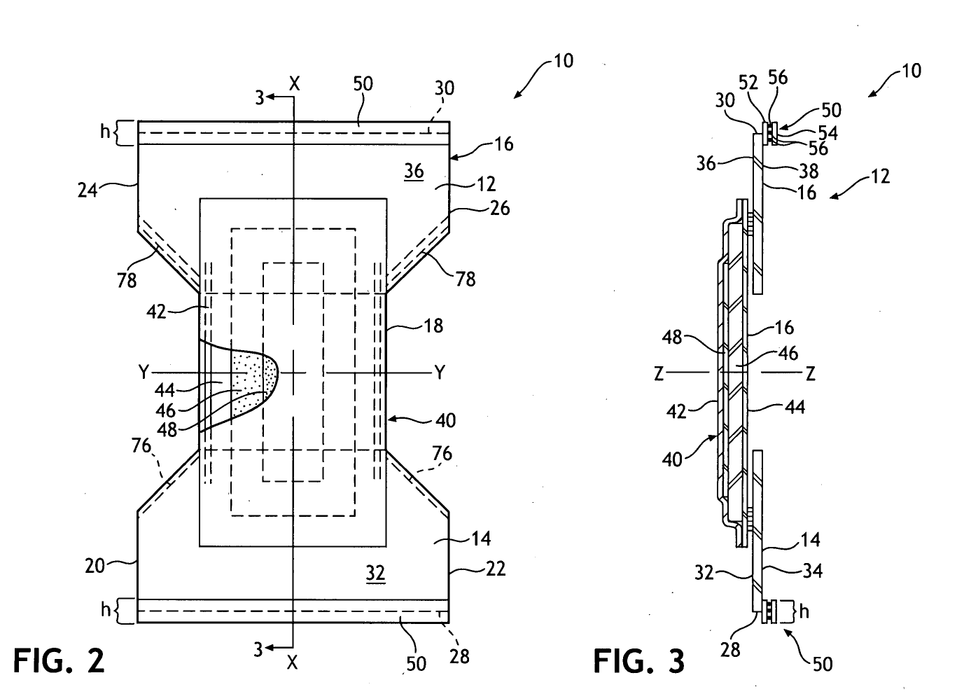

Referring to FIGS. 2 and 3, the disposable pant-like absorbent undergarment 10 is depicted in an open configuration solely for the purpose of better showing the various components. The open undergarment 10 has a longitudinal central axis X-X, a transverse central axis Y-Y and a vertical central axis Z-Z. The disposable pant-like absorbent undergarment 10 has a chassis 12 which includes a front region 14, a back region 16 and a crotch region 18. The crotch region 18 joins the front region 14 to the back region 16. The front and back regions, 14 and 16 respectively, are stretchable. By “stretchable” it is meant that the regions 14 and 16 can be increased in size, for example lengthened, widened or extended in one or more dimensions by applying a force, such as by pulling. The crotch region 18 can be stretchable or non-stretchable but desirably is non-stretchable. The stretchable front and back regions, 14 and 16 respectively, are also capable of being retracted or retracting to or towards their original pre-stretched dimensions. By “retracted or retracting” it is meant that the regions 14 and 16 can be reduced in size, for example made shorter in one or more dimensions by removing the force that was applied to stretch the region. As an example, a 1 inch (2.54 centimeters (cm)) strip of material can be stretched to about 2 inches (about 5 cm) and then when the stretching force is removed, the material will retract to or towards its original pre-stretched length of 1 inch (2.54 cm).

As noted above for the front and back regions, 14 and 16 respectively, if the crotch region 18 is stretchable then it will also be retractable. However, if the crotch region 18 is non-stretchable, it follows that it is also non-retractable.

The original width of the front region 14, measured parallel to the transverse central axis Y-Y, is measured as the distance between a first side edge 20 and a second side edge 22. The original width is measured before a force is applied to the front region 14. After being stretched and having the pulling force removed, the front region 14 will retract back to or towards the original width. The retracted dimension is measured after the force used to stretch the material has been removed. Likewise, the original width of the back region 16, measured parallel to the transverse central axis Y-Y, is measured as the distance between a first side edge 24 and a second side edge 26. A common way of making the front region 14 and the back region 16 stretchable is to form them from an elastic material. By “elastic” it is meant that the material has the ability to return to its initial dimension or size after deformation in a relatively short period of time. Examples of elastic materials include but are not limited to natural and synthetic rubbers, elastomeric films, some thermoplastic films, polyolefins such as polypropylene and polyethylene, laminates formed from two or more layers wherein at least one layer is elastic, as well as other material known to those skilled in the art.

Still referring to FIGS. 2 and 3, the front region 14 has a first end 28 and the back region 16 has a second end 30. The chassis 12 is folded approximately along the transverse central axis Y-Y such that the first end 28 aligns with the second end 30.

The front region 14 and the back region 16 are shown as being separate and discontinuous from one another although the front, back and crotch regions, 14, 16 and 18 respectively, could be formed from a single piece of material, if desired. Desirably, the front region 14 is formed from a similar or identical material as the back region 16. The front region 14 has an inner surface and an outer surface 34 (see FIG. 3). The inner or bodyside surface 30 will be in direct contact with the user's skin when the disposable pant-like absorbent undergarment 10 is worn. The outer or garment facing surface 32 will be spaced away from the user's skin and will be adjacent to any outer clothing that the user may be wearing. Likewise, the back region 16 has an inner surface 36 and an outer surface 38 (see FIG. 3). The inner or bodyside surface 36 will be in direct contact with the user's skin when the disposable pant-like absorbent undergarment 10 is worn. The outer or garment facing surface 38 will be spaced away from the user's skin and will be adjacent to any outer clothing that the user may be wearing.

The front and back regions, 14 and 16 respectively, can be constructed from various materials. The material can be a single layer or be a laminate of two or more layers. Spunbond is a material that works well for the front and back regions, 14 and 16 respectively. Spunbond is a nonwoven material that is capable of being stretched at least a minimum amount. Spunbond is manufactured and sold by Kimberly-Clark Corporation having an office at 401 North Lake Street, Neenah, Wis. 54956. A second material from which the front and back regions, 14 and 16 respectively, can be constructed is a stretch bonded laminate (SBL). Exemplary SBL materials are described in U.S. Pat. No. 4,720,415, which is hereby incorporated by reference and made a part hereof. A stretch bonded laminate can be formed from three layers wherein the elastic core or middle layer is elongated before the two outer nonwoven layers are attached. The attachment can be by adhesive, by heat, by pressure, by a combination of heat and pressure, etc. Another material option for the front and back regions, 14 and 16 respectively, is a necked bonded laminate (NBL). The necked bonded laminate is also a three layer laminate but the elastic core or middle layer is not pre-stretched prior to being attached to the two outer nonwoven layers. The outer layers are neck stretched before the elastic core or middle layer is attached to them. Exemplary NBL materials are described in U.S. Pat. No. 5,336,545, which is hereby incorporated by reference and made a part hereof. Other examples of elastomeric materials that can be used for the front and back regions, 14 and 16 respectively, include a continuous filament stretch bonded laminate (CFSBL), a vertical filament laminate (VFL), a necked stretch bonded laminate (NSBL) and a necked thermal laminate (NTL). Combinations of the above materials can also be used. Exemplary CFSBL materials are described in U. S. Pat. No. 5,385,775, which is hereby incorporated by reference and made a part hereof.

It should also be noted that the front and back regions, 14 and 16 respectively, can be constructed from an elastic film that is capable of being stretched in at least one direction and desirably in two directions. Desirably, the two directions correspond to the machine direction and cross-direction of the disposable pant-like absorbent undergarment 10. Alternatively, the front and back regions, 14 and 16 respectively, can be an elastic nonwoven that has a machine direction or a cross-direction stretch. Extensible materials can also be used along with various stretchable and/or elastic materials known to those skilled in the art.

Still, other options for the front and back region, 14 and 16 respectively, can include two outer layers having a plurality of elastic strands sandwiched therebetween. The elastic strands can be formed from LYCRA. LYCRA is a registered trademark of E. I. DuPont de Nemours & Company having an office at 1007 Market Street, Wilmington, Del. 19898. The elastic strands can be aligned approximately parallel to one another or be angled or skewed relative to one another. The elastic strands can vary in shape, size, configuration and/or length. The diameter and/or cross-sectional configuration of the elastic strands, the decitex (weight in grams per 10,000 meters of a strand) of the elastic strands, and the tension imparted into the elastic strands can all be varied to suit one's particular product needs. The elastic strands can have a round, semi-circular, square, rectangular, oval or some other geometrical configuration. The elastic strands can overlap, intersect or crisscross at least one other elastic strand. The various ways of positioning, orienting and/or adhering the elastic strands to the two outer layers are well known to those skilled in the art.

The disposable pant-like absorbent undergarment 10 also includes an absorbent assembly 40 present in said crotch region 18. The absorbent assembly 40 can include a liquid pervious bodyside liner 42, a liquid-impervious backsheet 44 and an absorbent 46 positioned therebetween. Alternatively, the absorbent assembly 40 can consist of one or more layers of an absorbent 46 without the bodyside liner 42 or the backsheet 44. The absorbent 46 in the absorbent assembly 40 can be formed from natural or synthetic materials. The absorbent 46 can be made from cellulosic fibers, wood pulp, textile fibers or from other absorbent materials known to those skilled in the art. Superabsorbents, commonly in solid form and in the shape of small particles, granules, flakes, etc., can be mixed, combined, attached, printed or otherwise added to the absorbent material to increase the absorbent capacity of the absorbent 46. A surge layer 48 can also be optionally used, which is normally positioned between the bodyside liner 42 and the absorbent 46. The surge layer 48 can function to rapidly acquire and temporarily retain body fluid, such as urine, before it can be absorbed into the absorbent 46. Desirably, the surge layer 48 is also capable of wicking body fluid lengthwise and/or widthwise across its surface as well as directing the body fluid downward in a z-direction, toward the absorbent 46.

Referring now to FIGS. 1-5, the disposable pant-like absorbent undergarment 10 further includes a waistband 50 secured to said first and second ends 28 and 30 of said front and back regions, 14 and 16 respectively. A portion of the waistband 50 overlaps each of the front and back regions, 14 and 16 respectively, and projects outward therefrom. The waistband 50 can be described as extending outward from the first and second ends 28 and 30 in a cantilevered configuration. The waistband 50 can have a height dimension h, measured parallel to the longitudinal central axis X-X, see FIGS. 2-5, which is at least about 0.75 inches (about 19 mm). Desirably, the height dimension h of the waistband 50 can range from between about 1 inch to about 2.5 inches (about 25 millimeters (mm) to about 64 mm). More desirably, the waistband 50 has a height dimension h of from between about 1 inch to about 2 inches (about 25 mm to about 51 mm). Even more desirably, the waistband 50 has a height dimension h of from between about 1.25 inches to about 1.75 inches (about 32 mm to about 45 mm). The waistband 50 can overlap the front and back regions, 14 and 16 respectively, by a distance of at least about 0.25 inches (about 6 mm). Desirably, the waistband 50 can overlap the front and back regions, 14 and 16 respectively, by a distance of from between about 0.25 inches to about 1.75 inches (about 6 mm to about 45 mm). Even more desirably, the waistband 50 can overlap the front and back regions, 14 and 16 respectively, by a distance of from between about 0.25 inches to about 1 inch (about 6 mm to about 25 mm).

The waistband 50 also extends out from and beyond the first and second ends 28 and 30 of the front and back regions, 14 and 16 respectively, by a distance of at least about 0.5 inches (about 13 mm). Desirably, the waistband 50 will extend out from and beyond the first and second ends 28 and 30 by a distance of from between about 0.5 inches to about 1.75 inches (about 13 mm to about 45 mm). More desirably, the waistband 50 will extend out from and beyond the first and second ends 28 and 30 by a distance of from between about 0.75 inches to about 1.5 inches (about 19 mm to about 38 mm).

The waistband 50 can be constructed from almost any elastic material having stretch and retraction capabilities. A desirable nonwoven material from which the waistband 50 can be constructed is spunbond. In FIG. 3, the waistband 50 is depicted as a laminate containing a first layer 52, a second layer 54 and two or more elastic strands 56 positioned therebetween. The outer two layers 52 and 54 can be constructed or formed from a woven or a nonwoven material, a natural or synthetic material, an elastic film, a thermoplastic film, or from any other material known to those skilled in the art. The number of elastic strands 56 positioned between the two outer layers 52 and 54 can vary depending upon the width of the waistband 50. For a waistband 50 having a width of less than about 2.5 inches (about 64 mm), about 2 to 12 elastic strands 56 can be employed. Desirably, the waistband 50 will utilize from between 2 to 10 elastic strands 56. More desirably, the waistband 50 will utilize from between 2 to 8 elastic strands 56. In FIGS. 3-5, two elastic strands 56 are depicted in the waistband 50.

The elastic strands 56 can be formed from LYCRA. The elastic strands 56 should extend along the entire length of the waistband 50 but could optionally be shorter in length, if desired. The elastic strands 56 should be positioned in an approximately parallel relationship to one another but could be arranged in an intersecting or crossing pattern, if desired. The elastic strands 56 should be spaced apart a predetermined distance depending upon how much tension, stretch and retraction is required for a particular product. A spacing distance between adjacent elastic strands 56 can range from between about 3 mm to about 10 mm. Desirably, the spacing distance between adjacent elastic strands 56 is from between about 5 mm to about 10 mm. The exact number of elastic strands 56, the decitex of each elastic strand 56, the exact distance adjacent elastic strands 56 are spaced apart, the method employed for adhering the elastic strands 56 to the outer layers 52 and 54, as well as the stretch, elongation and retraction capabilities of the elastic strands 56 will all dictate how many elastic strands 56 are needed. For example, a diaper having a narrow waistband may not need to have as much staying or holding power to retain the diaper around an infant's torso as a large adult incontinent undergarment designed to hold larger quantities of urine voided by the adult user. In addition, an adult wearing such an incontinent undergarment may be more mobile and upright for longer periods of time and therefore require a more secure waistband.

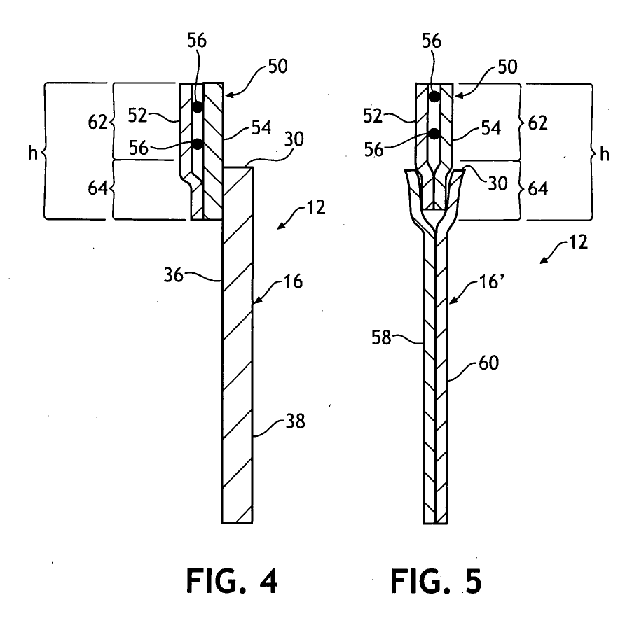

Turning our attention to FIGS. 3-5, the waistband 50 is shown being attached or secured to the chassis 12 three different ways. In FIG. 3, the waistband 50 is depicted as being secure to the outer surfaces 34 and 38 of the front and back regions, 14 and 16 respectively. In FIG. 4, the waistband 50 is depicted as being secure to the inner surface 36 of the back region 16. It should be noted that the front region 14 is not shown but the waistband 50 could be secured to the inner surface 32 of the front region 14 as well. In FIG. 5, the back region 16′ is depicted as being constructed from a two layer laminate having a first layer 58 and a second layer 60. The waistband 50 is shown being secured between the two layers 58 and 60.

It should also be noted that the chassis 12 can be formed from a laminate having three or more layers and the waistband 50 can be secured between any of such layers. In addition, it is also possible to secure the waistband 50 to a portion of the chassis 12 which is non-elastic (not shown). This non-elasticized region or zone can range in dimension from between about 0.25 inches to about 2 inches (about 6 mm to about 51 mm). The non-elasticized region or zone can be formed from two or more similar materials or from two or more dissimilar materials. The amount of overlap between the waistband 50 and the chassis 12 and the elastic properties of the overlapped members may be modified to alter the overall tension and fit of the disposable pant-like absorbent undergarment.

Referring to FIGS. 4 and 5, one will notice that the waistband 50 is depicted as having an elastic zone 62 and a non-elastic zone 64. The elastic zone 62 contains two or more elastic strands 56 while the non-elastic zone 64 does not contain any elastic strands 56. The non-elastic zone 64 is essentially non-elastic and is incapable of retracting on its own. Providing the first and second layers 52 and 54 are formed from a non-elastic material, the non-elastic zone is not capable of stretching or retracting except where it is secured to a chassis 12 that is formed from an elastic material. In FIGS. 4 and 5, the non-elastic zone 64 overlaps the back regions 16 and 16′ by a distance of at least 0.25 inches (about 6 mm). Likewise, even though not shown in FIGS. 4 and 5, the non-elastic zone 64 can overlap the front region 14 by a distance of at least 0.25 inches (about 6 mm). Desirably, the elastic zone 62 will have a height dimension, measured parallel to the longitudinal central axis X-X, that is equal to or greater than the non-elastic zone 64.

By contrast, it should be noted that the waistband 50 depicted in FIG. 3 does not have a non-elastic zone 64 but instead is constructed such that the entire height dimension is an elastic zone 62.

Turning back to FIG. 1, the disposable pant-like absorbent undergarment 10 further includes a pair of side seams 66 and 68 which function to join, bond and/or secure the front region 14 to the back region 16. The pair of side seams 66 and 68 extend through the waistband 50 as well to form a unitary undergarment. By “unitary” it is meant that the undergarment 10 is designed to be stepped into by a user and the undergarment 10 is then pulled up along the user's legs and thighs and positioned around the user's torso. There is no need to first open a unitary undergarment before it is applied to a user's body. In addition, if the pair of side seams 66 and 68 is torn or broken, the unitary undergarment essentially becomes unusable. The unitary disposable pant-like absorbent undergarment 10 has a longitudinal central axis X-X, a waist opening 70 and a pair of leg openings 72 and 74.

The disposable pant-like absorbent undergarment 10 further includes elastic members 76 and 78, each of which at least partially surrounds the pair of leg openings 72 and 74. The elastic member 76 can consist of one or more elastic strands secured to the front region 14 and the elastic member 78 can consist of one or more elastic strands secured to the back region 16. Each of the elastic members 76 and 78 can be formed as a continuous or a non-continuous member. In FIGS. 1 and 2, each of the elastic members 76 and 78 are depicted as two separate and distinct members that are spaced apart from one another. However, a single elastic member 76 and/or 78, consisting of 2 or 3 elastic strands, could be employed that extend from the side seam 66 to the opposite side seam 68. Such a continuous elastic member 76 and/or 78 would extend completely across the absorbent assembly 40.

Referring again to FIG. 1, when the disposable pant-like absorbent undergarment 10 is constructed as recited above, an undergarment more closely resembling cloth underwear is obtained. One reason for this is that the waistband 50 has a greater retracted length than the elastic members 76 and 78, and in turn the elastic members 76 and 78 have a greater retracted length than the remaining portion of the chassis 12. The remaining portion of the chassis 12 consists of the front and back regions, 14 and 16 respectively, located between the waistband 50 and the elastic members 76 and 78. In FIG. 1, the waistband 50 is denoted as “A”, the elastic members 76 and 78 are denoted as “B”, and the remainder of the chassis 12 is denoted as “C”. In short, the amount of retraction of each can be mathematically stated as:
A>B>C

It should be noted that the waistband 50 should have a retracted length “A” which is more than 5% in reduced dimension when compared to the retracted length of the chassis 12, denoted by “B”. Desirably, the waistband 50 should have a retracted length “A” which is more than 10% in reduced dimension when compared to the retracted length of the chassis 12, denoted by “B”. More desirably, the waistband 50 should have a retracted length “A” which is more than 15% in reduced dimension when compared to the retracted length of the chassis 12, denoted by “B”. Even more desirably, the waistband 50 should have a retracted length “A” which is more than 20% in reduced dimension when compared to the retracted length of the chassis 12, denoted by “B”.

Still referring to FIG. 1, another way of stating the above condition is to say that the waistband 50 will retract to a shorter length “L₁” than any other portion of the disposable pant-like absorbent undergarment 10. In physical terms, the waistband 50 should have a retracted length “L₁” which is at least about 0.5 inches (about 12 mm) less in dimension than the retracted length “L₂” of the remainder of the chassis 12, denoted as “C” and being the distance located between the waistband 50 and the elastic members 76 and 78, denoted as “B”. Desirably, the waistband 50, denoted by “A”, should have a retracted length “L₁” which is at least about 1 inch (about 25 mm) less in dimension than the retracted length “L₂” of the remainder of the chassis 12, denoted as “C” and being the distance located between the waistband 50 and the elastic members 76 and 78, denoted as “B”.

The waistband 50 is considered to be retracted when the top 1 inch (25.4 mm) of the waistband 50 located adjacent to the waist opening 70 retracts more than 1 inch (25.4 mm) when measured parallel to the transverse central axis Y-Y, as compared to the full width of the chassis 12, from side seam 66 to side seam 68, and excluding the absorbent assembly 40. The waistband 50 is also considered to be retracted when the top 1 inch (25.4 mm) of the waistband 50 located adjacent to the waist opening 70 retracts more than 1 inch (25.4 mm) when measured parallel to the transverse central axis Y-Y, as compared to the full width of the elastic members 76 or 78, from side seam 66 to side seam 68, and excluding the absorbent assembly 40.

When such a structure as described above is present, the waistband 50 will impart a convex configuration to the pair of side seams 66 and 68, relative to the longitudinal central axis X-X, before the disposable pant-like absorbent undergarment 10 is worn by a user. The front profile shown in FIG. 1 depicts the more underwear-like appearance once the undergarment 10 is removed from its package. The side seams 66 and 68 will exhibit a smooth curvature very similar to cloth underwear. In addition, the waistband 50 extends upward from the chassis 12 and this feature, along with its greater retraction length, creates a smaller waist opening 70 relative to the maximum width dimension of the chassis 12, as measured parallel to the transverse central axis Y-Y. The final configuration appears very similar to cloth underwear and provides the user with a peace of mind feeling. Aesthetically, it is more difficult for a third party to notice that the disposable pant-like absorbent undergarment 10 is not cloth underwear.

While the invention has been described in conjunction with several specific embodiments, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

We claim:

1. A disposable pant-like undergarment comprising:
a) a chassis having a stretchable front region, a stretchable back region, and a crotch region joining said front region to said back region, said front region having a first end and said back region having a second end, and said chassis being folded such that said first end aligns with said second end;
b) an absorbent assembly present in said crotch region;
c) a waistband secured to said first and second ends and projecting outward therefrom;
d) a pair of side seams joining said front region to said back region and extending through said waistband to form a pant-like undergarment having a longitudinal central axis, a waist opening and a pair of leg openings; and
e) elastic members at least partially surrounding said pair of leg openings, wherein said waistband has a greater retracted length than said elastic members and said elastic members have a greater retracted length than said chassis located between said waistband and said elastic members, and said waistband imparts a convex configuration to each of said pair of side seams relative to said longitudinal central axis before said undergarment is worn by a user.

2. The disposable pant-like undergarment of claim 1 wherein said stretchable front region and said stretchable back region are constructed from an identical material.

3. The disposable pant-like undergarment of claim 1 wherein said crotch region is not stretchable.

4. The disposable pant-like undergarment of claim 3 wherein said absorbent assembly includes a liquid permeable bodyside liner, a liquid-impermeable backsheet and an absorbent positioned therebetween, and said front region and said back regions are secured to said liquid-impermeable backsheet.

5. The disposable pant-like undergarment of claim 1 wherein said waistband has a retracted length which is more than about 5% in reduced dimension than said retracted length of said chassis located between said waistband and said elastic members.

6. The disposable pant-like undergarment of claim 5 wherein said waistband has a retracted length which is more than about 10% in reduced dimension than said retracted length of said chassis located between said waistband and said elastic members.

7. The disposable pant-like undergarment of claim 1 wherein said waistband has a retracted length which is at least 0.5 inches less in dimension than said retracted length of said chassis located between said waistband and said elastic members.

8. The disposable pant-like undergarment of claim 7 wherein said waistband has a retracted length which is at least 1 inch less in dimension than said retracted length of said chassis located between said waistband and said elastic members.

9. The disposable pant-like undergarment of claim 1 wherein said waistband has an elastic zone and a non-elastic zone and said non-elastic zone overlaps each of said front and back regions by a distance of at least about 0.25 inches.

10. A disposable pant-like undergarment comprising:
a) a chassis having a stretchable front region, a stretchable back region, and a crotch region joining said front region to said back region, said front region having a first end and said back region having a second end, and said chassis being folded such that said first end aligns with said second end;
b) an absorbent assembly present in said crotch region and extending into a portion of said front and back regions;
c) a waistband secured to said first and second ends and projecting outward therefrom;
d) a pair of side seams joining said front region to said back region and extending through said waistband to form a pant-like undergarment having a longitudinal central axis, a waist opening and a pair of leg openings; and
e) elastic members at least partially surrounding said pair of leg openings, wherein said waistband has a retracted length which is more than about 5% in reduced dimension than said elastic members, and said waistband imparts a convex configuration into each of said pair of side seams relative to said longitudinal central axis before said undergarment is worn by a user.

11. The disposable pant-like undergarment of claim 10 wherein each of said front and back regions has an outer surface and said waistband is secured to said outer surfaces.

12. The disposable pant-like undergarment of claim 10 wherein each of said front and back regions has an inner surface and said waistband is secured to said inner surfaces.

13. The disposable pant-like undergarment of claim 10 wherein each of said front and back regions is a two layer laminate and said waistband is secured between said two layers.

14. The disposable pant-like undergarment of claim 10 wherein each of said pair of side seams has a curvature spanning a length of at least about 5 inches.

15. The disposable pant-like undergarment of claim 10 wherein said waistband has an elastic zone and a non-elastic zone, and said non-elastic zone overlaps each of said front and back regions by a distance of at least about 0.25 inches.

16. A disposable pant-like undergarment comprising:
a) a chassis having a stretchable front region, a stretchable back region, and a crotch region joining said front region to said back region, said front region having a first end and said back region having a second end, and said chassis being folded such that said first end aligns with said second end;
b) an absorbent assembly present in said crotch region into a portion of said front and back and extending regions;
c) a waistband secured to said first and second ends and projecting outward therefrom;
d) a pair of side seams joining said front region to said back region and extending through said waistband to form a pant-like undergarment having a longitudinal central axis, a waist opening and a pair of leg openings; and
e) elastic members at least partially surrounding said pair of leg openings, wherein said waistband has a retracted length which is more than about 10% in reduced dimension than said elastic members and said elastic members have a greater retracted length than said chassis located between said waistband and said elastic members, and said waistband imparts a convex configuration into each of said pair of side seams relative to said longitudinal central axis before said undergarment is worn by a user.

17. The disposable pant-like undergarment of claim 16 wherein said waistband contains from between 2 to 12 spaced apart, elastic strands and has a width of at least about 1 inch.

18. The disposable pant-like undergarment of claim 16 wherein said waistband has an elastic zone and a non-elastic zone, and said non-elastic zone overlaps each of said front and back regions by a distance of at least about 0.25 inches.

19. The disposable pant-like undergarment of claim 18 wherein said elastic zone has a greater height dimension than said non-elastic zone.

20. The disposable pant-like undergarment of claim 18 wherein said non-elastic zone is incapable of retracting on its own.
